(19) United States
(12) Patent Application Publication
(10) Pub. No.: US 2018/0104231 A1
(43) Pub. Date: Apr. 19, 2018

(54) BENZALKONIUM CHLORIDE FREE OPHTHALMIC COMPOSITION CONTAINING (+)-(S)-4-[4-CHLOROPHENYL) (2-PYRIDYL) METHOXY] PIPERDINO] BUTYRIC ACID OR PHARMACOLOGICALLY ACCEPTABLE ACID ADDITION SALT, THEREOF

(71) Applicant: INTERA HEALTHCARE PLC, Andhra Pradesh (IN)

(72) Inventor: Hari Raghu Ram Desu, Andhra Pradesh (IN)

(21) Appl. No.: 15/730,575

(22) Filed: Oct. 11, 2017

Related U.S. Application Data
(60) Provisional application No. 62/408,623, filed on Oct. 14, 2016.

(51) Int. Cl. A61K 31/4545; A61K 47/02; A61K 9/00

(52) U.S. Cl. CPC A61K 31/4545; A61K 9/0048; A61K 47/02

(57) ABSTRACT

The present invention relates to pharmaceutical composition(s) containing active ingredient and inactive ingredients without preservative(s). The pharmaceutical composition contains an active ingredient for treating allergic conjunctivitis, spring catarrh, pollinosis, and the like. The pharmaceutical composition is packaged in unit-of-use or multi-dose containers. The claimed composition reduces side effects associated with multiple administration of eye-drops containing preservatives.

### CROSS REFERENCE TO RELATED APPLICATION

This application claims priority to U.S. Provisional Patent Application No. 62/408,623 filed Oct. 14, 2016, the entire content of which is incorporated herein by reference in its entirety.

### FIELD

The present invention relates to preservative-free ophthalmic composition containing Bepotastine Besylate, and other inactive ingredients. Bepotastine Besylate use is for treating allergic conjunctivitis, spring catarrh, pollinosis, and the like.

### BACKGROUND

The present invention relates to benzenesulfonic acid salt or benzoic acid salt of (S)-4-[4-[(4-chlorophenyl)(2-pyridyl)methoxy]piperidino]butanoic acid or (+)-(S)-4-[4-chlorophenyl) (2-pyridyl) methoxy] piperdino] butyric acid monobenzenesulfonate which have excellent antihistaminic activity and antiallergic activity.

As described in Japanese Provisional Patent Publication, Kita, Fujiwara et al., 2000 (JP 2000198784), the piperdine derivative has characteristics, such as stimulation or suppression on the central nerves, whose actions are equivalent to treating allergic conjunctivitis, allergic rhinitis, pollinosis, and spring catarrh.

U.S. Pat. No. 6,780,877 filed on 12 Sep. 2001 teaches the acid addition salt of (S)-4-[4-[(4-chlorophenyl)(2-pyridyl)methoxy]piperidino]butanoic acid which has little hygroscopicity and excellent in physicochemical stability so that it is particularly suitable compound as a medicine. Also, said document teaches a medical composition containing the compound as an effective ingredient.

A piperdine derivative is being marketed as an effective therapeutic agent for treating allergies in humans. In particular, a tablet (Talion®) comprising (+)-(S)-4-[4-chlorophenyl)(2-pyridyl)methoxy]piperdino]butyric acid monobenzenesulfonate (general name: Bepotastine Besilate) is marketed for treating allergenic rhinitis and itching associated with hives and dermatoses. An ophthalmic solution (Bepreve®) containing (+)-(S)-4-[4-chlorophenyl)(2-pyridyl)methoxy]piperdino]butyric acid monobenzenesulfonate is marketed for treating allergic conjunctivitis, pollinosis, and spring catarrh.

However, bepotastine besilate and pharmacologically acceptable acid addition salt thereof are unstable to light in an aqueous solution, and colored or precipitated with the lapse of time. Such instability of active in light makes the use thereof as an aqueous liquid preparation difficult. In the case of an aqueous liquid preparation such as an eye drop, it is necessary to block light by preserving in a light-shielding container.

U.S. Pat. No. 2,929,274 discloses a method comprising adding boric acid and/or borax and glycerin, but according to this method, stabilization of (+)-(S)-4-[4-[(4-chlorophenyl)(2-pyridyl)methoxy]piperidino]butyric acid and a pharmacologically acceptable acid addition salt thereof to light was not observed. As a general stabilization method, a method comprising placing in the coexistence of an antioxidant such as BHT etc., and the like are known (JP-A-7-304670).

U.S. Pat. No. 8,784,789 filed on 30 Jul. 2003 and U.S. Pat. No. 8,877,168 filed on 25 Jun. 2014 disclose aqueous liquid preparation containing (+)-(S)-4-[4-[(4-chlorophenyl)(2-pyridyl)methoxy]piperidino]butyric acid or a pharmacologically acceptable acid addition salt thereof, which is stabilized with a water-soluble metal chloride. The acid addition salts of (+)-(S)-4-[4-[(4-chlorophenyl)(2-pyridyl)methoxy]piperidino]butyric acid taught by said document is monobenzenesulfonate. Further, the metal chloride is at least one kind selected from sodium chloride, potassium chloride and calcium chloride. Benzalkonium chloride is present in the preparation as preservative. Use of preservatives and other stabilizing agent have several side effects such as irritation in eyes.

Further as a general rule, ophthalmic products are packaged in multiple-use containers. Preservatives are used to prevent bacterial contamination in multiple-use ophthalmic preparations. One or more preservatives and ancillary agents may be chosen from, benzalkonium chloride, benzyl alcohol, chlorobutanol, parabens, phenylmercuric acetate, stabilized oxychloro complexes (otherwise known as Plurite®), and thimerosal. Typically, preservatives are used in the concentration range, 0.0001% w/v to 1.0% w/v. If no preservative is desired, compositions may be sterile packaged in unit-of-use containers or unpreserved sterility assured multiple-dose containers. As per the art described in Bepreve® package insert, ophthalmic composition may need to be administered one drop into the affected eye(s) twice a day. Henceforth, ophthalmic composition necessitates the inclusion of preservative to prevent microbial contamination during multiple-use.

Bepotastine Besilate ophthalmic solution (Bepreve®) has benzalkonium chloride (BAC) at 0.005% w/v. Benzalkonium chloride belongs to class, cationic surfactants. Gasset, 1977; Green, Hull et al., 1977; Burstein, 1980; Collin, 1986 reported the undesirable side effects of cationic surfactants at even lower concentrations. These reports elucidated physiological changes (edema) and ultra-structural changes to corneal epithelium and endothelium upon exposure of BAC, 0.0001% even for a minimum of 15 min. At clinical concentration (0.005%), BAC caused cell wrinkling to lifting of corneal epithelial cells. On BAC exposure, microvilli were often lost at the edges of the corneal epithelial cells. According to Nichols, microvilli along with microplicae provide structural framework that supports and binds a complex of related factors including tears, mucus, and immunoglobulins that have the common function of protecting the eye. Filamentous cell coats (glycocalyx) extend from microvilli surfaces. And these filamentous projections form scaffolding which is believed to be mucus, with its content of immunoglobulins by weak chemical interactions to the epithelial surface. Loss of microvilli upon BAC exposure (multiple instillations) could aggravate the symptoms associated with allergic conjunctivitis, spring catarrh, pollinosis, and the like.

In view of the afore-said, there is a need for a ophthalmic composition of (+)-(S)-4-[4-chlorophenyl)(2-pyridyl)methoxy]piperdino]butyric acid monobenzenesulfonate (Bepreve®) which is free of benzalkonium chloride so that such BAC-free composition could protect structural and physiological aspects of microvilli. Miller, 2008; Leonardi, Bogacka et al., 2012 reported allergen clearance mechanism of mucus and immunoglobulin secretions from ocular surfaces. Thus, BAC-free ophthalmic composition could protect the anti-allergic action of (+)-(S)-4-[4-chlorophenyl)(2-pyridyl)methoxy]piperdino]butyric acid monobenzenesulfonate.

### OBJECT OF THE INVENTION

The primary object of the present invention is to provide a preservative-free ophthalmic composition containing (+)-(S)-4-[4-chlorophenyl)(2-pyridyl)methoxy]piperdino]butyric acid or a pharmacologically acceptable acid addition salt thereof (Bepotastine Besylate), and other inactive ingredients.

The further object of the present invention is to provide benzalkonium chloride (BAC) free ophthalmic composition containing Bepotastine Besylate, and other inactive ingredients.

Another object of the present invention is to provide a stable preservative free ophthalmic composition containing (+)-(S)-4-[4-chlorophenyl)(2-pyridyl)methoxy]piperdino]butyric acid or a pharmacologically acceptable acid addition salt thereof.

Another object of the present invention is to provide a stable BAC-free ophthalmic composition of (+)-(S)-4-[4-chlorophenyl)(2-pyridyl)methoxy]piperdino]butyric acid monobenzenesulfonate)(Bepreve®).

The further object of the present invention is to provide the method of preparation of preservative-free ophthalmic composition containing (+)-(S)-4-[4-chlorophenyl)(2-pyridyl)methoxy]piperdino]butyric acid or a pharmacologically acceptable acid addition salt thereof (Bepotastine Besylate), and other inactive ingredients.

### SUMMARY OF THE INVENTION

In order to achieve the afore-said objectives, the present invention provide preservative-free ophthalmic pharmaceutical composition comprising:
(a) (+)-(S)-4-[4-chlorophenyl)(2-pyridyl)methoxy]piperdino]butyric acid or a pharmacologically acceptable acid addition salt thereof;
(b) water-soluble metal chloride;
(c) sodium dihydrogen phosphate;
(d) sodium hydroxide;
(e) disodium edetate optionally;
(f) sterile water for injection;
(g) pH of the composition in the range, 6-8.

According to the one aspect of the invention the active ingredient of the afore-said composition is a (+)-(S)-4-[4-chlorophenyl)(2-pyridyl)methoxy]piperdino]butyric acid or a pharmacologically acceptable acid addition salt thereof, and has a concentration in the range, with a lower limit of 0.2% w/v and an upper limit of 2.0% w/v.

According to the another aspect of the invention the acid addition salt in the present pharmaceutical composition is monobenzenesulfonate, and has a concentration in the range, with a lower limit of 0.2% w/v and an upper limit of 2.0% w/v.

According to the another aspect of the invention the water-soluble metal chloride is at least one selected from sodium chloride, potassium chloride, and calcium chloride and has a concentration in the range, with a lower limit of 0.1% w/v and an upper limit of 1.5% w/v. Preferably the water-soluble metal chloride is sodium chloride, and has a concentration in the range, with a lower limit of 0.1% w/v and an upper limit of 1.5% w/v.

According to the another aspect of the invention the buffer is phosphate buffer, and has a concentration in the range, with a lower limit of 0.1% w/v and an upper limit of 10.0% w/v. Preferably, the buffer agent is sodium dihydrogen phosphate buffer, and has a concentration in the range, with a lower limit of 0.1% w/v and an upper limit of 10.0% w/v.

According to another aspect of the invention the pH of the composition is in the range of 6.0-8.0.

According to the another aspect of the invention the pharmaceutical composition of the present invention is an eye-drop.

According to the another aspect of the invention a method of preparing preservative-free pharmaceutical composition, which comprises;
i) (+)-(S)-4-[4-chlorophenyl)(2-pyridyl)methoxy]piperdino]butyric acid monobenzenesulfonate,
ii) water-soluble metal chloride,
iii) sodium dihydrogen phosphate,
iv) sodium hydroxide,
v) disodium edetate optionally, and
vi) sterile water for injection, and pH 6-8.

According to another aspect of the invention a method of packaging preservative-free pharmaceutical composition of the present invention in unit-of-use or sterility assured multi-dose containers.

According to the another aspect of the invention a method of flushing preservative-free pharmaceutical composition of the present invention with insert gas selected at least one from Helium, Nitrogen, Argon, or Neon, and the like.

### DETAILED DESCRIPTION

The following presents a detailed description of various aspects of the present invention. The aspects of the present invention are described in detail with reference to the examples. However, the present subject matter is not limited to these aspects which are only provided to explain more clearly the present subject matter to a person skilled in the art of the present disclosure.

The specification may refer to “an”, “one”, “different” or “some” aspect(s) in several locations. This does not necessarily imply that each such reference is to the same aspect(s), or that the feature only applies to a single aspect. Single features of different aspects may also be combined to provide other aspects.

As used herein, the singular forms “a”, “an” and “the” are intended to include the plural forms as well, unless expressly stated otherwise. It will be further understood that the terms “includes”, “comprises”, “including” and/or “comprising” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations and arrangements of one or more of the associated listed items.

Pharmaceutical composition described herein is an aqueous preparation containing (+)-(S)-4-[4-chlorophenyl) (2-pyridyl) methoxy] piperdino] butyric acid (represented in Formula I) or a pharmacologically acceptable acid addition salt (e.g. monobenzenesulfonate represented in Formula II) thereof. Preferably, the present invention is related to preservative-free compositions of (+)-(S)-4-[4-chlorophenyl)(2-pyridyl)methoxy]piperdino]butyric acid or a pharmacologically acceptable acid addition salt. More preferably, the present invention relates to preservative-free pharmaceutical composition of (+)-(S)-4-[4-chlorophenyl)(2-pyridyl)methoxy]piperdino]butyric acid monobenzenesulfonate.

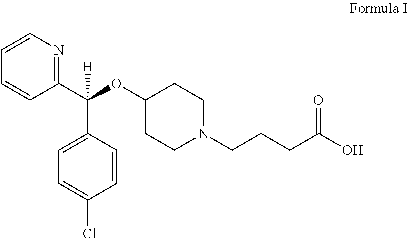 [SMILES: O=C(O)CCCN1CCC(O[C@@H](c2ccc(Cl)cc2)c2ccccn2)CC1]

* indicates an asymmetric carbon, which contributes to anti-histaminic and anti-allergenic activities.

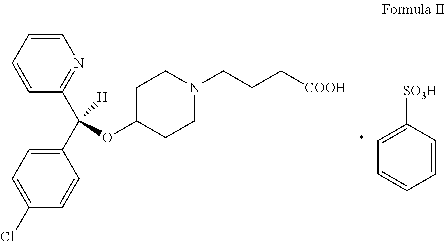 [SMILES: O=C(O)CCCN1CCC(O[C@@H](c2ccc(Cl)cc2)c2ccccn2)CC1 ; O=S(=O)(O)c1ccccc1]

For demonstration of proof-concept studies, various experimental studies are being conducted in vitro to show that the existing preservative containing (+)-(S)-4-[4-chlorophenyl)(2-pyridyl)methoxy]piperdino]butyric acid and a pharmacologically acceptable acid addition salt can be superseded with preservative-free ophthalmic composition(s) containing (+)-(S)-4-[4-chlorophenyl)(2-pyridyl)methoxy]piperdino]butyric acid or a pharmacologically acceptable acid addition salt thereof.

In accordance with main aspect of the present invention, is provided preservative-free ophthalmic pharmaceutical composition comprising:
(h) (+)-(S)-4-[4-chlorophenyl)(2-pyridyl)methoxy]piperdino]butyric acid or a pharmacologically acceptable acid addition salt thereof;
(i) water-soluble metal chloride;
(j) sodium dihydrogen phosphate;
(k) sodium hydroxide;
(l) disodium edetate optionally;
(m) sterile water for injection;
(n) pH of the composition in the range, 6-8.

According to an aspect of the present invention, the concentration of (+)-(S)-4-[4-chlorophenyl)(2-pyridyl)methoxy]piperdino]butyric acid or a pharmacologically acceptable salt thereof as monobenzenesulfonate is a lower limit of 0.2% w/v, preferably a lower limit of 0.5% w/v and an upper limit of 2.0% w/v, preferably an upper limit of about 1.5% w/v. Preferably, the concentration of active ingredient, (+)-(S)-4-[4-chlorophenyl)(2-pyridyl)methoxy]piperdino]butyric acid or a pharmacologically acceptable salt thereof as monobenzenesulfonate is based on the art of package information, Bepreve®.

As described in the U.S. patent, Kita, Fujiwara et al., 2001, acid addition salt of (+)-(S)-4-[4-chlorophenyl)(2-pyridyl)methoxy]piperdino]butyric acid can be, for example, salts with hydrohalic acid such as HCl, HBr and the like; organic acid salts such as acetate, benzoate, benzenesulfonate, cinnamate, citrate, cyclohexylsulfamate, dihydroxyfumarate, ethanesulfonate, fumarate, hydroxyacetate, malate, maleate, malonate, methanesulfonate, oxalate, propionate, pyruvate, p-toluenesulfonate, salicylate, succinate, 2-hydroxypropionate, 4-aminosalicylate and the like. Preferably, acid addition salt is benzenesulfonate and benzoate. More preferably, acid addition salt is monobenzenesulfonate. The concentration of monobenzenesulfonate in the composition is in the range, with a lower limit of 0.2% w/v and an upper limit of 2.0% w/v.

According to an aspect of the present invention, light-stabilizing agents such as water-soluble metal chlorides are used to prevent light-triggered color change and precipitate formation in pharmaceutical compositions containing (+)-(S)-4-[4-chlorophenyl)(2-pyridyl)methoxy]piperdino]butyric acid or a pharmacologically acceptable acid addition salt. Preferably, water-soluble metal chloride belongs to alkali metal chlorides such as sodium chloride, potassium chloride and the like; and alkaline earth metal chlorides such as calcium chloride and the like; and can be used either alone or in combination. Preferably, water-soluble metal chloride is sodium chloride.

According to an aspect of the present invention, the concentration of water-soluble metal chloride is a lower limit of 0.1% w/v and an upper limit of 1.5% w/v, preferably a lower limit of 0.2% w/v and an upper limit of 1.2% w/v. In particular, water-soluble chloride is sodium chloride, whose lower limit of concentration is 0.1% w/v and an upper limit of 1.2% w/v. Potassium chloride as water-soluble metal chloride, the lower limit of concentration is 0.1% w/v and an upper limit of 1.0% w/v, whereas calcium chloride as water-soluble metal chloride, the lower limit of concentration is 0.1% w/v and an upper limit of 1.5% w/v.

According to an aspect of the present invention, as a general rule, tonicity of pharmaceutical compositions administered to eye is either adjusted to hypertonicity, isotonicity, or hypotonicity relative to tear fluids. Stabilizing agents can also function as tonicity agents. As described in U.S. patent, Higashiyama, 2003, the concentration of water-soluble metal chlorides is considered as appropriate within the above-mentioned concentration range, such that the osmotic pressure is in the range, 230-350 mOsm. And the concentration of water-soluble metal chloride is in the range, 0.1% w/v to 2.0% w/v.

According to an aspect of the present invention, besides the above-mentioned additives, buffers may be added to pharmaceutical compositions to maintain the pH of composition at a particular value. In this invention, buffers, such as acetate buffer, amino acid, borate buffer, citrate buffer, phosphate buffer, tartrate buffer, and the like can be used to stabilize pH of pharmaceutical compositions. Preferably, buffer used in the present invention is a phosphate buffer. More preferably, buffer used in this invention is a sodium dihydrogen phosphate buffer. The concentration of buffer agent is in the range, with a lower limit of 0.1% w/v and an upper limit of 10.0% w/v; preferably, a lower limit of 0.1% w/v and an upper limit of 5% w/v; more preferably, a lower limit of 0.1% w/v and an upper limit of 2.5% w/v.

According to an aspect of the present invention, the pH of pharmaceutical composition(s) is adjusted to not less than about 4, 5, 6, and not more than about 8.5. Preferably, the pH of pharmaceutical composition described in the invention is in the range, 6-8. More preferably, the pH of pharmaceutical composition(s) is in the range, 6.5-7.5. The pH of the present invention is adjusted with alkalis or acids and the like. Preferably, pH of pharmaceutical composition is adjusted to basic pH with sodium hydroxide or pH of pharmaceutical composition is adjusted to acidic pH with acidic agents, such as hydrochloric acid and phosphoric acid. The concentration of pH adjusting agents, such as alkalis or acids is in the range, 0.01% v/v to 10% v/v. Preferably, a lower limit of 0.001% v/v and an upper limit of 5% v/v; more preferably, a lower limit of 0.001% v/v and an upper limit of 2.5% v/v.

According to an aspect of the present invention, as described in U.S. patent, Xia, Salamone et al., 2006, chelating or sequestration agents are added to minimize binding of metal ions with lens and protein deposits. If not removed of these deposits, eventually settle on lens and blur vision. Therefore, chelating agents are added to ophthalmic compositions. In general, chelating agents are added in the concentration range, a lower limit of 0.01% w/v and an upper limit of 0.2% w/v.

According to an aspect of the present invention, pharmaceutical compositions can be administered as an eye drop, a nasal drop, an ear drop and the like. Preferably, the pharmaceutical compositions are administered to humans and animals. More preferably, the pharmaceutical compositions are administered to humans. Particularly, the pharmaceutical composition is administered to humans for treating allergic conjunctivitis, spring catarrh, pollinosis, and the like. The pharmaceutical compositions are instilled one drop into the eyes of affected patients. These eye-drops are instilled 2 times-a-day. The frequency of administration of eye-drops is based on the degree/intensity of disease symptoms.

According to an aspect of the present invention, pharmaceutical composition can be produced by a production method known per se, such as a method described in the literature, Hecht, 2006; Desu, Narang et al., 2013. In brief, (+)-(S)-4-[4-chlorophenyl)(2-pyridyl)methoxy]piperdino]butyric acid or a pharmacologically acceptable acid addition salt is added to an aliquot of sterile water for injection containing light-stabilizing water-soluble metal chloride. Pharmaceutical composition is buffered to a specific pH value using phosphate buffer salt. Preferably, the buffer salt is sodium dihydrogen phosphate. And, pH of the pharmaceutical composition is finely adjusted using either sodium hydroxide or hydrochloric acid/phosphoric acid. The pharmaceutical composition is subjected to sterile filtration and aseptically filled into unit-of-use or multi-dose containers. Aseptic filling of the pharmaceutical composition is performed under inert gas flush. The filled-up containers are aseptically sealed to prevent microbial contamination.

### Examples

The present invention will now be explained with the help of the following example, however; the scope of the invention should not be limited to said example. It is to be understood that the above described aspects are merely illustrative principles of the present invention and that many variations may be devised by those skilled in the art without departing from the scope of the present invention. It is, therefore, intended that such variations be included with the scope of the claims.

The present invention is explained in detail through some examples citing pharmaceutical compositions of (+)-(S)-4-[4-chlorophenyl)(2-pyridyl)methoxy]piperdino]butyric acid or a pharmacologically acceptable salt thereof as monobenzenesulfonate.

TABLE 1
Eye Drop Formulations

| | Formu- | Formu- | Formu- | Formu- | Formu- |
|---|---|---|---|---|---|
| *Ingredient (% w/v) | la 1 | la 2 | la 3 | la 4 | la 5 |
| Bepotastine Besilate | 1.5 | 1.5 | 1.5 | 1.5 | 1.5 |
| Sodium chloride | 0.60 | 0.65 | 0.68 | 0.72 | 0.76 |
| Sodium dihydrogen | 0.1 | 0.1 | 0.1 | 0.1 | 0.1 |
| phosphate monohydrate | | | | | |
| Sodium hydroxide | q.s. | q.s. | q.s. | q.s. | q.s. |
| Water for injection | q.s. | q.s. | q.s. | q.s. | q.s. |
| pH | 6.8 | 6.8 | 6.8 | 6.8 | 6.8 |

*Ingredient compositions are expressed in terms of % w/v

TABLE 2
Eye Drop Formulations

| | Formu- | Formu- | Formu- | Formu- | Formu- |
|---|---|---|---|---|---|
| *Ingredient (% w/v) | la 6 | la 7 | la 8 | la 9 | la 10 |
| Bepotastine Besilate | 1.5 | 1.5 | 1.5 | 1.5 | 1.5 |
| Sodium chloride | 0.45 | 0.50 | 0.55 | 0.60 | 0.65 |
| Sodium dihydrogen | 0.2 | 0.2 | 0.2 | 0.2 | 0.2 |
| phosphate monohydrate | | | | | |
| Sodium hydroxide | q.s. | q.s. | q.s. | q.s. | q.s. |
| Water for injection | q.s. | q.s. | q.s. | q.s. | q.s. |
| pH | 6.8 | 6.8 | 6.8 | 6.8 | 6.8 |

*Ingredient compositions are expressed in terms of % w/v

The present compositions of Bepotastine Besilate without benzalkonium chloride as a preservative may be marketed in multiple dose containers. As a result, novel formulation results in the same bioavailability of Bepotastine Besilate in the eye without the unwanted side effects associated with benzalkonium chloride. These side effects range from hyperemia, blepharitis, corneal erosion, depression, epiphoria, eye discharge, eye dryness, eyelid edema, eyelid erythema and eyelid pruritus.

The present invention manifests preparation of benzalkonium chloride free Bepotastine Besilate eye drop formulations. These formulation compositions could be as listed in Table 1 and Table 2, but not limited to the table listed formulations. Product composition is prepared using sterile vessels in clean rooms. In brief, Bepotastine Besilate ophthalmic solution is manufactured as: a) product vehicle, i.e., water for injection (WFI) is poured into jacketed vessel kept at a defined temperature, b) WFI is kept under stirring condition, c) inactive ingredients are added to vehicle, d) active ingredient (Bepotastine Besilate) is added to composition, e) pH of solution is adjusted with sodium hydroxide, f) remaining WFI is added to make solution volume to final volume, g) Bepotastine Besilate solution is subjected to sterile filtration, h) sterile solution is filled into multi-dose ophthalmic dispensing containers and sealed, and i) product containers are stored at 15-25° C.

The present invention embodies testing of benzalkonium chloride free Bepotastine Besilate eye drop formulations against culture media for aerobic and anaerobic organisms. Two drops of preservative free Bepotastine Besilate formulations were dispersed onto media plates containing Soybean-Casein Digest agar and Sabouraud Dextrose agar, in sterile environmental conditions. Soybean-Casein Digest agar media plates were incubated at 30-35° C. for 24 hours to detect the presence of Escherichia coli, Pseudomonas aeruginosa and Staphylococcus aureus. While Sabouraud Dextrose agar media plates were incubated at 20-25° C. for 48 hours to detect for Candida albicans and 20-25° C. for 7 days for the presence of Aspergillus niger.

TABLE 3
Detection of Microbial Contamination in Eye Drop
Formulations (Initial and Six-Month Stored Samples)

| | Formulations | | | | |
|---|---|---|---|---|---|
| | Formu- | Formu- | Formu- | Formu- | Formu- |
| Microbe | la 1 | la 2 | la 3 | la 4 | la 5 |
| Escherichia coli | ND | ND | ND | ND | ND |
| Pseudomonas aeruginosa | ND | ND | ND | ND | ND |
| Staphylococcus aureus | ND | ND | ND | ND | ND |
| Candida albicans | ND | ND | ND | ND | ND |
| Aspergillus niger | ND | ND | ND | ND | ND |

ND denotes not detected in media culture plates. These formulations, but not limited to the above were stored for initial (zero hour) and six months storage conditions at 15-25° C. Initial and six months stored samples were tested for the presence of microbial organisms.

Absence of microbial organisms in media indicates that the multiple-dose containers or ophthalmic sequeeze dispensers used for the dispensing of eye drops protected the formulation contents from the growth of microbial organisms.

What is claimed is:

1. A preservative free pharmaceutical composition comprising:
a) (+)-(S)-4-[4-chlorophenyl)(2-pyridyl)methoxy]piperdino]butyric acid or a pharmacologically acceptable acid addition salt thereof,
b) a water-soluble metal chloride,
c) a buffer agent,
d) alkali metal hydroxide,
e) pH of the composition in the range, 6-8, and
f) sterile water.

2. The composition as claimed in claim 1, wherein the acid addition salt is (+)-(S)-4-[4-chlorophenyl)(2-pyridyl)methoxy]piperdino]butyric acid monobenzenesulfonate.

3. The composition as claimed in claim 1, wherein the acid addition salt has a concentration in the range, with lower limit of 0.2% w/v and an upper limit of 2.0% w/v.

4. The composition as claimed in claim 1, wherein the water-soluble metal chloride is sodium chloride, potassium chloride or calcium chloride.

5. The composition as claimed in claim 4, wherein the water-soluble metal chloride is sodium chloride.

6. The composition as claimed in claim 1, wherein the water-soluble metal chloride has a concentration in the range, with lower limit of 0.1% w/v and an upper limit of 1.2% w/v.

7. The composition as claimed in claim 1, wherein the buffer agent is sodium dihydrogen phosphate monohydrate.

8. The composition as claimed in claim 1, wherein the buffer agent has a concentration in the range, with lower limit of 0.1% w/v and an upper limit of 2.0% w/v.

9. The composition as claimed in claim 1, wherein the pharmaceutical composition has a pH in the range, 6 to 8.0.

10. The composition as claimed in claim 1, wherein the alkali metal hydroxide is sodium hydroxide.

11. The composition as claimed in claim 1, wherein the alkali metal hydroxide has a concentration appropriate to adjust the pH of pharmaceutical composition to 6-8.

12. The composition as claimed in claim 1, wherein the pharmaceutical composition is an eye-drop.

13. A preservative free pharmaceutical composition comprising:
(a) active ingredient comprising (+)-(S)-4-[4-chlorophenyl)(2-pyridyl)methoxy]piperdino]butyric acid or a pharmacologically acceptable acid addition salt thereof;
(b) a water-soluble metal chloride;
(c) sodium dihydrogen phosphate monohydrate;
(d) sodium hydroxide, and
e) sterile water;
wherein the pharmacologically acceptable acid addition salt has a concentration selected from the range of a lower limit concentration of 0.2% w/v and an upper limit concentration of about 1.5% w/v.

14. A preservative free pharmaceutical composition comprising:
(a) active ingredient comprising (+)-(S)-4-[4-chlorophenyl)(2-pyridyl)methoxy]piperdino]butyric acid or a pharmacologically acceptable acid addition salt thereof;
(b) a water-soluble metal chloride;
(c) sodium dihydrogen phosphate monohydrate;
(d) sodium hydroxide, and
e) sterile water;
wherein the pharmaceutical composition is aseptically stored in multi-dose dispensing container.

15. A preservative free pharmaceutical composition comprising:
(a) active ingredient comprising (+)-(S)-4-[4-chlorophenyl)(2-pyridyl)methoxy]piperdino]butyric acid or a pharmacologically acceptable acid addition salt thereof;
(b) a water-soluble metal chloride;
(c) sodium dihydrogen phosphate monohydrate;
(d) sodium hydroxide, and
(e) sterile water;
wherein the pharmaceutical composition is provided in a multi-dose dispensing container suitable for administration into eyes of patients.
